(19) United States
(12) Patent Application Publication
(10) Pub. No.: US 2026/0077047 A1
(43) Pub. Date: Mar. 19, 2026

(54) CANCER THERAPY

(71) Applicant: TURBINE SIMULATED CELL TECHNOLOGIES LTD, London (GB)

(72) Inventors: Boldizsar Frigyes ELEK, London (GB); Magdolna DJUREC; Daniel VERES; Iván FEKETE; Dorottya LUKÁCS-NÉMETH, London (GB); Dorina Viktória SIMON, London (GB)

(73) Assignee: TURBINE SIMULATED CELL TECHNOLOGIES LTD, London (GB)

(21) Appl. No.: 19/109,029

(22) PCT Filed: Sep. 5, 2023
(86) PCT No.: PCT/GB2023/052293

§ 371 Date: Mar. 5, 2025

(30) Foreign Application Priority Data
Sep. 5, 2022 (GB) 2212918.3

(51) Int. Cl. A61K 45/06; A61K 31/4545; A61P 35/00

(52) U.S. Cl. CPC A61K 45/06; A61K 31/4545; A61P 35/00

(57) ABSTRACT

A pharmaceutical composition comprising an NEK1 inhibitor and uses thereof in the treatment of cancer.

This application relates to a pharmaceutical composition comprising an NEK1 inhibitor and uses thereof in the treatment of various cancers. This application also relates to pharmaceutical combination products comprising an NEK1 inhibitor and an ATM inhibitor and uses thereof.

### BACKGROUND OF THE INVENTION

Treating cancer and cancers that show drug resistance is a significant problem leading to the suffering and deaths of millions worldwide.

For example, it is estimated that over a million new cases of cancer will be diagnosed each year in the United States alone, and over half a million people in the US will die from the disease each year.

The most common cancers include head and neck cancer, oral cancer, breast cancer, lung and bronchus cancer, prostate cancer, colon and rectum cancer, melanoma of the skin, bladder cancer, non-Hodgkin lymphoma, kidney and renal pelvis cancer, endometrial cancer, leukaemia, pancreatic cancer, thyroid cancer and liver cancer.

While various treatments are available, there remains a need in the art for improved solutions to the problem of treating cancer and drug resistant cancer.

### SUMMARY OF THE INVENTION

In a first aspect of the invention, there is provided a pharmaceutical composition for use in the treatment of cancer, wherein the composition comprises a NEK1 inhibitor.

In an embodiment, the cancer is head and neck cancer. In an embodiment, the cancer is oral cancer. In an embodiment, the cancer is mantle cell lymphoma. In an embodiment, the cancer is melanoma.

In a second aspect of the invention there is provided a pharmaceutical combination for use in the treatment of cancer comprising an NEK1 inhibitor and one or more additional therapeutic agents in synergistically effective amounts for simultaneous, separate or sequential administration to a subject in need thereof, wherein the one or more additional therapeutic agents comprise an ataxia telangiectasia mutated (ATM) kinase inhibitor

In an embodiment, the ATM inhibitor is one or more of AZD1390, AZ32, KU-55933, KU60019, KY12420, Torin2 or ETP46464.

In an embodiment, the ATM inhibitor is (AZD1390).

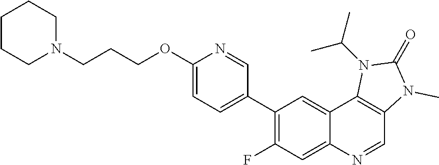 [SMILES: CC(C)n1c(=O)n(C)c2cnc3cc(F)c(-c4ccc(OCCCN5CCCCC5)nc4)cc3c21]

In an embodiment, the ATM inhibitor is (AZ32).

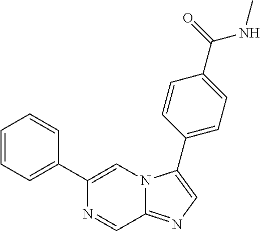 [SMILES: CNC(=O)c1ccc(-c2cnc3cnc(-c4ccccc4)cn23)cc1]

In an embodiment, the ATM inhibitor is (KU-55933).

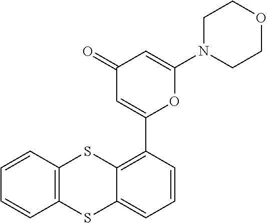 [SMILES: O=c1cc(-c2cccc3c2Sc2ccccc2S3)oc(N2CCOCC2)c1]

In an embodiment, the ATM inhibitor is (KU60019).

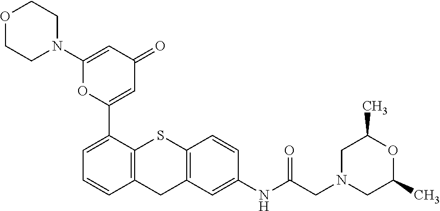 [SMILES: C[C@@H]1CN(CC(=O)Nc2ccc3c(c2)Cc2cccc(-c4cc(=O)cc(N5CCOCC5)o4)c2S3)C[C@H](C)O1]

In an embodiment, the ATM inhibitor is (KY12420).

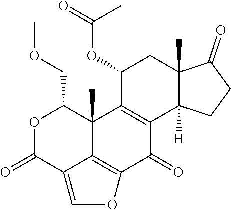 [SMILES: COC[C@H]1OC(=O)c2coc3c2[C@@]1(C)C1=C(C3=O)[C@@H]2CCC(=O)[C@@]2(C)C[C@H]1OC(C)=O]

In an embodiment, the ATM inhibitor is (Torin2).

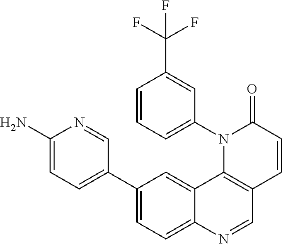 [SMILES: Nc1ccc(-c2ccc3ncc4ccc(=O)n(-c5cccc(C(F)(F)F)c5)c4c3c2)cn1]

In an embodiment, the ATM inhibitor is (ETP46464).

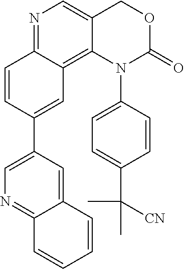 [SMILES: CC(C)(C#N)c1ccc(N2C(=O)OCc3cnc4ccc(-c5cnc6ccccc6c5)cc4c32)cc1]

In an embodiment the cancer is breast cancer.

In an embodiment the cancer is oral cancer.

In an embodiment the cancer exhibits a p53 mutation and a homologous recombination (HR) mutation.

In an embodiment the cancer exhibits a p53 mutation and a homologous recombination (HR) deficiency.

In an embodiment the cancer does not exhibit an ATM mutation.

The invention makes use of a combination of two inhibitor components in order to impair effective DNA repair, and so to target cancer cells. Each inhibitor acting synergistically in order to prevent cancer cells from proliferating, and/or killing the cancer cells. The combination of the invention has been found to be more effective than the predicted sum of each component additively. This synergistic interaction makes the combination more effective against cancers. This synergy can lead to greater activity, and/or to lower doses of the active components. Beneficially, lower doses of the active components can reduce side effects and can save on costs. Additionally, it is believed that drug resistant cancers may be more effectively treated, in particular cancers resistant to monotherapy. The combinations of inhibitors were intelligently identified by computer modelling.

It is believed that a combination of the deficiencies in the expression of two or more genes may lead to cell death. The deficiencies may be mutations, epigenetic alterations, and/or the inhibition of gene products. Cancer cells, which due to the mutations they possess, may therefore be sensitised to the inhibition of certain gene products over healthy tissue. The second aspect of the invention makes use of this sensitivity.

Herein disclosed there is provided a pharmaceutical combination comprising the combination according to the second aspect of the invention, and/or the embodiments thereof, for use as a medicament.

In an embodiment, the pharmaceutical combination is for use in the treatment of cancer, wherein the cancer is selected from breast cancer, head and neck cancer, oral cancer, mantle cell lymphoma or melanoma cancer.

In an embodiment, the pharmaceutical combination is for use in the treatment of breast cancer. In an embodiment, the pharmaceutical combination is for use in the treatment of head and neck cancer. In an embodiment, the pharmaceutical combination is for use in the treatment of oral cancer. In an embodiment, the pharmaceutical combination is for use in the treatment of mantle cell lymphoma. In an embodiment, the pharmaceutical combination is for use in the treatment of melanoma, wherein the melanoma cancer is selected from superficial spreading melanoma, nodular melanoma, lentigo maligna melanoma, acral lentiginous melanoma, ocular melanoma, mucosal lentiginous melanoma, demoplastic melanoma, amelonotic melanoma, cutaneous melanoma or anorectal melanoma.

In an embodiment a ‘synergistically effective amount’ provides a positive ‘Excess Over Bliss’ (EOB) value. EOB can be defined as the measured kill-rate of Drug A at a certain dosage in combination with Drug B at a certain dosage, subtracted by the theoretical additive kill-rate of both drugs as monotherapies. EOB values vary between −1 and 1 where a negative value indicates antagonism at a given dose pair, and a positive value indicates synergism at a given dose pair. Therefore, synergy is an interaction between two agents that causes the total effect (e.g. cancer cell killing activity) to be greater than the sum of the individual effects of each agent. In an embodiment a synergistically effective amount may be determined by an alternative method of determining synergy such as the highest single agent (HSA) model.

In an embodiment, the pharmaceutical combination is for use in the treatment of cancer, wherein the combination is formulated for oral administration or intravenous administration.

Herein disclosed, there is provided a pharmaceutical combination comprising the combination according to the second aspect of the invention, and/or the embodiments thereof, for the manufacture of a medicament for the treatment of cancer, and optionally drug resistant cancer.

In an embodiment the ratio of the NEK1 inhibitor to the ataxia telangiectasia mutated (ATM) kinase inhibitor in the pharmaceutical composition is equal or less than 1000:1, 500:1, 100:1, 50:1, 20:1, 10:1, 5:1, 2:1, 1.5:1, 1:1, 1:1.5, 1:2, 1:5, 1:10, 1:20, 1:50, 1:100, 1:500, 1:1000

In a third aspect of the invention is provided a pharmaceutical composition for use in the treatment of a cancer in a patient wherein the cancer exhibits a p53 mutation, wherein the composition comprises an NEK1 inhibitor.

In an embodiment the cancer additionally exhibits a homologous recombination (HR) mutation.

In an embodiment the cancer additionally exhibits a homologous recombination (HR) deficiency.

In an embodiment the composition of the first or third aspect comprises 0.05 mg to 100 mg of an NEK1 inhibitor.

In an embodiment the composition of the second aspect comprises 0.05 mg to 100 mg of an NEK1 inhibitor.

In an embodiment the composition of the first or third aspect comprises 0.5 mg to 50 mg of an NEK1 inhibitor.

In an embodiment the composition of the second aspect comprises 0.5 mg to 50 mg of an NEK1 inhibitor.

In an embodiment the composition of the second aspect comprises 0.05 mg to 100 mg of an ATM inhibitor.

In an embodiment the composition of the second aspect comprises 0.1 mg to 50 mg of an ATM inhibitor.

In an embodiment the composition of the second aspect comprises 0.5 mg to 25 mg of an ATM inhibitor.

Therapeutic dosages may be varied depending upon the requirements of the patient, the severity of the condition being treated, and the compound being employed. The magnitude of an effective dose of a compound will vary with the nature of the severity of the condition to be treated and with the particular compound and its route of administration. The daily dose range may be from about 10 μg to about 30 mg per kg body weight of a human and non-human animal, optionally from about 50 μg to about 30 mg per kg of body weight of a human and non-human animal, for example from about 50 μg to about 10 mg per kg of body weight of a human and non-human animal, for example from about 100 μg to about 30 mg per kg of body weight of a human and non-human animal, for example from about 100 μg to about 10 mg per kg of body weight of a human and non-human animal and most preferably from about 100 μg to about 1 mg per kg of body weight of a human and non-human animal.

In a fourth aspect of the invention is provided a pharmaceutical composition comprising an NEK1 inhibitor and an ATM inhibitor

In an embodiment there is provided a pharmaceutical combination according to any aspect of the invention, and/or the embodiments thereof, together with at least one pharmaceutically acceptable excipient.

In an embodiment the combination is formulated as a tablet. In an embodiment the combination is formulated as a capsule. In an embodiment the combination is formulated for injection. In an embodiment the injection is intra-venous or subcutaneous. In an embodiment the combination is supplied in a sterile buffer solution or as a solid which can be suspended or dissolved in sterile buffer for injection. In an embodiment the pharmaceutically acceptable excipient(s) may be selected from, for example, carriers (e.g. a solid, liquid or semi-solid carrier), adjuvants, diluents (e.g. solid diluents such as fillers or bulking agents; and liquid diluents such as solvents and co-solvents), granulating agents, binders, flow aids, coating agents, release-controlling agents (e.g. release retarding or delaying polymers or waxes), binding agents, disintegrants, buffering agents, lubricants, preservatives, anti-fungal and antibacterial agents, antioxidants, buffering agents, tonicity-adjusting agents, thickening agents, flavouring agents, sweeteners, pigments, plasticizers, taste masking agents, stabilisers or any other excipients conventionally used in pharmaceutical compositions.

The term “pharmaceutically acceptable” as used herein means compounds, materials, compositions, and/or dosage forms which are, within the scope of sound medical judgment, suitable for use in contact with the tissues of a subject (e.g. a human subject) without excessive toxicity, irritation, allergic response, or other problem or complication, commensurate with a reasonable benefit/risk ratio. Each excipient must also be “acceptable” in the sense of being compatible with the other ingredients of the formulation.

The pharmaceutical combination may be a pharmaceutical composition which can be in any form suitable for oral, parenteral, topical, intranasal, intrabronchial, sublingual, ophthalmic, otic, rectal, intra-vaginal, or transdermal administration. Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches. Tablet compositions can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known in the art. Tablets may be designed to release the drug either upon contact with stomach fluids (immediate release tablets) or to release in a controlled manner (controlled release tablets) over a prolonged period of time or with a specific region of the GI tract.

A pharmaceutical composition typically comprises from approximately 1% (w/w) to approximately 95%, optionally % (w/w) active ingredients and from 99% (w/w) to 5% (w/w) of a pharmaceutically acceptable excipient (for example as defined above) or combination of such excipients. Preferably, the compositions comprise from approximately 20% (w/w) to approximately 90% (w/w) active ingredients and from 80% (w/w) to 10% of a pharmaceutically excipient or combination of excipients. The pharmaceutical compositions may comprise from approximately 1% to approximately 95%, optionally from approximately 20% to approximately 90%, active ingredients. Pharmaceutical compositions according to the invention may be, for example, in unit dose form, such as in the form of ampoules, vials, suppositories, pre-filled syringes, dragées, powders, tablets or capsules.

Tablets and capsules may contain, for example, 0-20% disintegrants, 0-5% lubricants, 0-5% flow aids and/or 0-99% (w/w) fillers/or bulking agents (depending on drug dose). They may also contain 0-10% (w/w) polymer binders, 0-5% (w/w) antioxidants, 0-5% (w/w) pigments. Slow release tablets would in addition typically contain 0-99% (w/w) release-controlling (e.g. delaying) polymers (depending on dose). The film coats of the tablet or capsule typically contain 0-10% (w/w) polymers, 0-3% (w/w) pigments, and/or 0-2% (w/w) plasticizers. Parenteral formulations typically contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils. The pharmaceutical formulation may be presented to a patient in “patient packs” containing an entire course of treatment in a single package, usually a blister pack. The pharmaceutical combination of the invention will generally be presented in unit dosage form and, as such, will typically contain sufficient compounds to provide a desired level of biological activity. For example, a formulation may contain from 1 nanogram to 2 grams of the active ingredients, e.g. from 1 nanogram to 2 milligrams of active ingredients. Within these ranges, particular sub-ranges of compound are 0.1 milligrams to 2 grams of active ingredients (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredients). For oral compositions, a unit dosage form may contain from 1 milligram to 2 grams, more typically 10 milligrams to 1 gram, for example 50 milligrams to 1 gram, e.g. 100 milligrams to 1 gram, of active ingredients. The active ingredients will be administered to a patient in need thereof (for example a human or animal patient) in an amount sufficient to achieve the desired therapeutic effect (effective amount). The precise amounts of compound administered may be determined by a supervising physician in accordance with standard procedures.

Various further aspects and embodiments of the present invention will be apparent to those skilled in the art in view of the present disclosure.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

### BRIEF DESCRIPTION OF THE FIGURES

The invention will now be further and more particularly described, by way of example only, and with reference to the accompanying drawings, in which:

FIG. 1A shows Western blotting of HSC-2 cells±treatment with NEK-1 sgRNA lentiviral vectors. Cells were collected 7 days and 10 days post infection.

FIG. 1B shows Western blotting of Daoy cells±treatment with NEK-1 sgRNA lentiviral vectors. Cells were collected 7 days and 10 days post infection.

FIG. 1C shows Western blotting of SK-MEL-24 cells±treatment with NEK-1 sgRNA lentiviral vectors. Cells were collected 10 days post infection.

FIG. 1D shows Western blotting of HCC1806 cells±treatment with NEK-1 sgRNA lentiviral vectors. Cells were collected 7 days post infection and followed by WB.

FIG. 1E shows Western blotting of NCI-H1563±treatment with NEK-1 sgRNA lentiviral vectors. Cells were collected 25 days post infection.

FIG. 1F shows Western blotting of Granta 519 cells treated with inducible NEK-1 sgRNA lentiviral vectors. Cells were grown for 7 days post infection in the presence or absence of 1 mg/mL doxycycline with a refresh of the medium on day 4.

FIG. 2A shows the ratio of GFP sgRNA transfected HSC-2 cells to mCherry-sgNT transfected cells over 21 days growth as determined by FACS analysis.

FIG. 2B shows the ratio of GFP sgRNA transfected Daoy cells to mCherry-sgNT transfected cells over 21 days growth as determined by FACS analysis.

FIG. 2C shows the ratio of GFP sgRNA transfected SK-MEL-24 cells to mCherry-sgNT transfected cells over 28 days growth as determined by FACS analysis.

FIG. 2D shows the ratio of GFP sgRNA transfected HCC1806 cells to mCherry-sgNT transfected cells over 28 days growth as determined by FACS analysis.

FIG. 3A shows the effect of NEK-1 knockout on proliferation of HSC-2 cells.

FIG. 3B shows the effect of NEK-1 knockout on proliferation of Daoy cells.

FIG. 3C shows the effect of NEK-1 knockout on proliferation of SK-MEL-24 cells.

FIG. 3D shows the effect of NEK-1 knockout on proliferation of HCC1806 cells.

FIG. 3E shows the effect of inducing NEK-1 knockout using sg1 on proliferation of Granta 519 cells.

FIG. 3F shows the effect of inducing NEK-1 knockout using sg2 on proliferation of Granta 519 cells.

FIG. 3G shows the effect of inducing NEK-1 knockout using sg3 on proliferation of Granta 519 cells.

FIG. 3H shows the effect of inducing NEK-1 knockout using sg4 on proliferation of Granta 519 cells.

FIG. 4A shows the effect of AZD1390 concentration on proliferation rate of HSC-2 cells±NEK-1 knockout. Error bars show standard deviation (n=3). Fits to IC50 behaviour are presented as lines with IC50 values given.

FIG. 4B shows the effect of AZD1390 concentration on proliferation rate of Daoy cells±NEK-1 knockout. Error bars show standard deviation (n=3). Fits to IC50 behaviour are presented as lines with IC50 values given.

FIG. 4C shows the effect of AZD1390 concentration on proliferation rate of HCC1806 cells±NEK-1 knockout. Error bars show standard deviation (n=3). Fits to IC50 behaviour are presented as lines with IC50 values given.

FIG. 4D shows the effect of AZD1390 concentration on proliferation rate of NCI-1563 cells±NEK-1 knockout. Error bars show standard deviation (n=3). Fits to IC50 behaviour are presented as lines with IC50 values given.

FIG. 5A shows the effect of inducible NEK-1 knockout on HSC-2 tumour growth. Error bars show standard deviation (n=10 or 15).

FIG. 5B shows the gene expression of NEK-1 in inducible HSC-2 NT and NEK-1 KO-sg2 tumour cells quantified by qPCR (n=5). Data is normalised to GAPDH expression.

FIG. 5C shows the effect of inducible NEK-1 knockout on Daoy tumour growth. Error bars show standard deviation (n=10 or 13).

FIG. 5D shows the gene expression of NEK-1 in inducible Daoy NT and NEK-1 KO-sg2 tumour cells quantified by qPCR (n=5). Data is normalised to GAPDH expression.

FIG. 5E shows the effect of constitutive NEK-1 knockout on HSC-2 tumour growth. Error bars show standard deviation (n=10).

FIG. 5F shows the expression of NEK-1 in constitutive HSC-2 NT and NEK-1 KO-sg2 tumour cells quantified by Western blotting (n=5). Data is normalised to beta-actin expression.

### DETAILED DESCRIPTION OF THE INVENTION

In a first aspect of the invention there is provided a pharmaceutical composition for use in the treatment of head and neck cancer, mantle cell lymphoma or melanoma, wherein the composition comprises an NEK1 inhibitor.

In an embodiment, the composition is for use in the treatment of head and neck cancer. In an embodiment, the composition is for use in the treatment of oral cancer. In an embodiment, the composition is for use in the treatment of mantle cell lymphoma. In an embodiment, the composition is for use in the treatment of melanoma.

In a further aspect of the invention, there is provided a pharmaceutical combination for use in the treatment of cancer comprising an NEK1 inhibitor and one or more additional therapeutic agents in synergistically effective amounts for simultaneous, separate or sequential administration to a subject in need thereof, wherein the one or more additional therapeutic agents comprise a ataxia telangiectasia mutated (ATM) kinase inhibitor.

In an embodiment, the ATM kinase inhibitor is one or more of AZD1390, AZ32, KU-55933, KU60019, KY12420, Torin2 or ETP46464. In an embodiment, the ATM kinase inhibitor is AZD1390.

In an embodiment, the cancer is breast cancer. In an embodiment, the cancer is oral cancer. In an embodiment, the cancer exhibits a p53 mutation and a homologous recombination (HR) mutation. In an embodiment, the cancer exhibits a p53 mutation and a homologous recombination (HR) deficiency. In an embodiment, the cancer exhibits an ATM mutation.

In an embodiment, the ratio of the NEK1 inhibitor to the ataxia telangiectasia mutated (ATM) kinase inhibitor in the pharmaceutical combination is equal or less than 1000:1, 500:1, 100:1, 50:1, 20:1, 10:1, 5:1, 2:1, 1.5:1, 1:1, 1:1.5, 1:2, 1:5, 1:10, 1:20, 1:50, 1:100, 1:500, 1:1000.

In a further aspect there is provided a pharmaceutical composition for use in the treatment of a cancer in a patient wherein the cancer exhibits a p53 mutation, wherein the composition comprises an NEK1 inhibitor. In an embodiment, the cancer additionally exhibits a homologous recombination (HR) mutation. In an embodiment, the cancer additionally exhibits a homologous recombination (HR) deficiency.

In an embodiment, the composition comprises 0.05 mg to 100 mg of an NEK1 inhibitor. In an embodiment, the composition or combination comprises 0.05 mg to 100 mg of an NEK1 inhibitor. In an embodiment, the composition comprises 0.5 mg to 50 mg of an NEK1 inhibitor.

In an embodiment, the combination comprises 0.5 mg to 50 mg of an NEK1 inhibitor. In an embodiment, the combination comprises 0.05 mg to 100 mg of an ATM inhibitor. In an embodiment, the combination comprises 0.1 mg to 50 mg of an ATM inhibitor. In an embodiment, the combination comprises 0.5 mg to 25 mg of an ATM inhibitor.

In a further aspect there is provided a pharmaceutical composition comprising an NEK1 inhibitor and an ATM inhibitor.

### METHODS AND EXAMPLES

### Example 1—Production of NEK1 Knockout Cell Line Strains

### 1a—sqRNA Design and Construction

Genomic information of NEK1 gene was obtained from NCBI website. Broad Institute sgRNA Designer (http://www.broadinstitute.org/rnai/public/analysis-tools/sgrna-design) was used to design sgRNAs of targeting sequences. The sgRNA sequences were respectively cloned into LentiCRISPRv2-GFP, LentiCRISPRv2-puro and pRSGT16-U6Tet-puro vectors.

TABLE 1
sequences encoding sgRNA for generating NEK-1 knockouts.

| | | Protospacer | | On Target |
|---|---|---|---|---|
| | | adjacent | | Efficacy |
| Name | sgRNA Sequence | motif | Direction | Score |
| NT | TGAGGATCATGTCGAGCGCC | Not | Not | Not |
| | (SEQ ID NO. 1) | applicable | applicable | applicable |
| sg1 | ATAACTGAGACACCAAACTG | CGG | antisense | 0.7599 |
| | (SEQ ID NO. 2) | | | |
| sg2 | AAGGAAAAAGAACTTACCTG | AGG | antisense | 0.69 |
| | (SEQ ID NO. 3) | | | |
| sg3 | GAAGTGAAGAGGCTGACATG | AGG | sense | 0.7059 |
| | (SEQ ID NO. 4) | | | |
| sg4 | GGAAGTGGTGAAGTAAAGGT | AGG | sense | 0.6256 |
| | (SEQ ID NO. 5) | | | |

1b—Lentiviral Packaging

For each transfection reaction (10 cm dish), DNA-Lipofectamine 2000 complexes were prepared:
i. In a 15 mL tube, the plasmid DNA (5 μg target plasmid, 3.75 μg psPAX2, 2.5 μg pVSG) was prepared in 1.5 mL of Opti-MEM medium.
ii. In a separate 15 mL tube, 33.75 μL Lipofectamine 2000 was diluted into 3 mL of Opti-MEM, mixed by pipetting and incubated for 5 minutes at room temperature.
iii. The DNA and the diluted Lipofectamine 2000 solutions were combined and mixed gently by pipetting, then incubated for 20 minutes at room temperature.
iv. 293FT cells were typsinized and counted. Cells were resuspended at a density of 1×10⁶ cells/ml in growth medium containing serum.
v. 10 mL of the 293FT cell suspension (10×10⁶ total cells) were added into 10 cm dishes. There were no antibiotics added to the medium.
vi. The DNA-Lipofectamine 2000 complexes were added to the cell suspensions. The cells were rocked back and forth to mix and incubated overnight at 37° C. in a CO₂ incubator.
vii. The next day, the media was removed and replaced with 10 mL of complete culture medium containing sodium pyruvate.
viii. Virus-containing supernatants were harvested after 48˜72 hours. supernatants were then centrifuged at 3000 rpm for 15 minutes at 4° C. to pellet cell debris.
ix. Viral supernatants were pipetted into cryovials in 1 ml aliquots and stored as viral stocks at −80° C.
   1c—Cell Line Culturing

HSC-2 and Daoy cells were cultured in EMEM medium supplemented with 10% Fetal Bovine Serum and 1% Antibiotic Antimycotic. SKMES1 cells were cultured in EMEM medium supplemented with 20% Fetal Bovine Serum and 1% Antibiotic Antimycotic. HCC1806 and NCIH1563 cells were cultured in RPMI-1640 medium supplemented with 10% Fetal Bovine Serum and 1% Antibiotic Antimycotic. Standard growth conditions were 37° C. with 5% CO₂.

Cells medium was refreshed every 3-4 days and cells were passaged twice every week.

Before use, for all parental and engineered cell lines, 5 ml supernatant of culturing cells was collected and centrifuged at 200 g for 5 minutes at room temperature. Then 1 ml was transferred to 1.5 ml EP tube for MP test using MycoAlert™ PLUS Mycoplasma Detection Kit according to manufacturer's protocol.

1d—Transfection of Target Cell Lines

### Day 1: Seeding for Viral Infection

i. Parental cells were removed from culture medium and washed once with PBS.
ii. Trypsin was added to 0.05% w/v and cells were incubated under standard conditions until they detached.
iii. Cells were diluted 2-fold with growth medium (see 1c) to stop trypsinization
iv. Cells were centrifuged at 200×g for 5 min at room temperature. The supernatant was removed and the cells were resuspended and counted using trypan blue.
v. 1˜3×10⁵ cells were seeded per well of the required number of 6-well plates, including an uninfected parental cells condition.
vi. Cells were incubated under standard conditions.

### Day 2: Viral Infection

vii. Culture medium was removed and replaced with 1 ml of viral stock (NT, NEK1-sg1/2/3, or HEK293FT growth medium for parental condition).
viii. 1 ul of 6 ug/ul polybrene was added to the 1 ml of virus-containing medium in each well to give a final polybrene concentration of 6 ug/ml.
ix. Cells were incubated overnight under standard conditions.

### Day 3: Removing the Virus

x. Virus-containing medium was removed and 2 ml of growth medium (see 1c) was added to each well for cell recovery.
x. Cells were incubated under standard conditions for 3 days to recover.

### Day 6: Recovery Day 3

xii. Growth medium was removed from each well and cells were washed once with PBS.
xiii. Trypsin was added to 0.05% w/v and cells were incubated under standard conditions until they detached.
xiv. 1 ml of complete growth medium was added to stop trypsinization.
xv. 1000 μL of cells were transferred to sterile 1.5 ml Eppendorf tubes and counted using trypan blue.
xvi. 1-2×10⁵ cells, depending on growth rate, from each of the single infection conditions were seeded back into complete culture medium and incubated under standard conditions for Western Blot analysis (See Example 2).
xvii. The same number of cells (1-2×10⁵ cells) from mCherry sgNT condition were mixed with cells from each of the GFP sgRNA conditions for competition assays (See Example 3).

### Example 2—Knockdown Analysis by Western Blotting

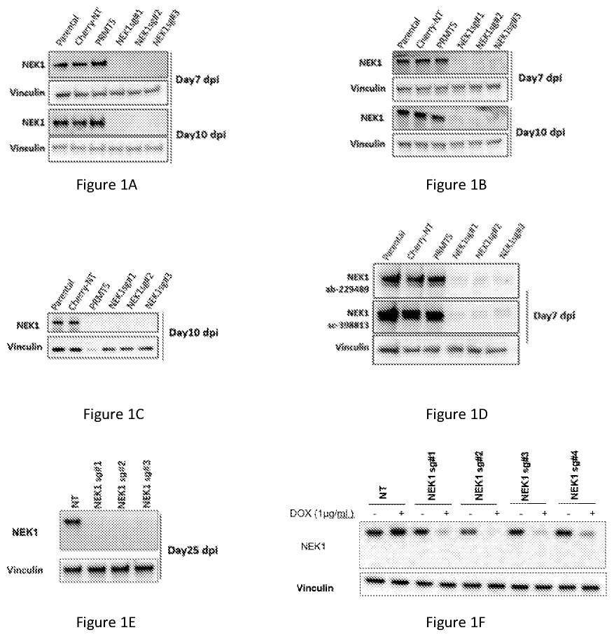

To demonstrate the successful knocking out of NEK1 on the target cells lines HSC-2, Daoy, SK-MEL-24, HCC1806 and NCI-H1563, the unmixed, single samples of infected cells were grown in complete culture medium (section 1c) under standard conditions for 7-10 days post infection. Analysis of the knockdown of target gene(s) by western blotting was carried out using standard protocols. The data presented in FIGS. 1A-E demonstrates that cells treated with sg #1-3 do not express significant quantities of NEK1, whereas cells treated with mCherry-sgNT or PRMT5 knockout strains express NEK1 to similar levels as the parental, non-infected, cells.

### Example 3—NEK1 Knockout Competition Assay

The effect of knocking out NEK1 was investigated using a competition assay against mCherry sgNT transfected HSC-2, Daoy, SK-MEL-24 and HCC1806 cells. The same number of cells (see Table 2) from mCherry sgNT condition were mixed with cells from each of the GFP sgRNA conditions in PBS buffer and seeded into 6-well plates for continued culturing at room temperature.

TABLE 2
Total cells seeded per cell line on Day 0 of competition
assay. Total is made up of 50% mCherry sg-NT infected
cells and 50% GFP sgRNA infected cells.

| Parental Cell Line | Total cells seeded on Day 0 |
|---|---|
| HSC-2 | 2 × 10⁵ |
| Daoy | 1 × 10⁵ |
| SK-MEL-24 | 2 × 10⁵ |
| HCC1806 | 2 × 10⁵ |

The remaining cells were analysed by FACS:
a. Cells were centrifuged at 200×g for 5 minutes at room temperature.
b. The supernatant was discarded and replaced with PBS. Cells were then centrifuged again.
c. Cells were resuspended in 300 μl-500 μl of PBS, depending on cell number, to reach 500˜1000 cells/ul and transferred into FACS tubes for analysis.

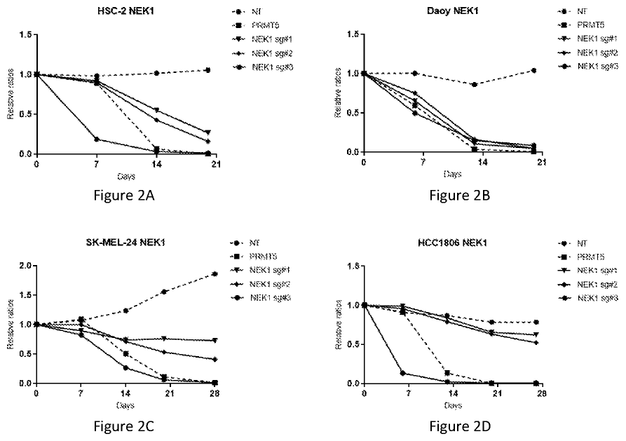

Further FACS analysis was carried out on the cells after 7, 14, 21 and in some cases 28 days. Data presented in FIGS. 2A-D reveal that NEK1 knockout is deleterious to growth in most cell lines. Achilles scores for each cell line are presented in Table 3

TABLE 3
Achilles scores for NEK1 knockout in each of the tested cell lines

| Parental Cell Line | Achilles score for NEK1 knockout |
|---|---|
| HSC-2 | −0.26 |
| Daoy | −0.731 |
| SK-MEL-24 | −0.174 |
| HCC1806 | 0.0382 |

### Example 4a—Cell Proliferation Assay—Constitutive Knockout

i. Cells were obtained from suspension or by trypsinising adhered cells then centrifuged at 200 g for 5 minutes at room temperature (RT).
ii. Cell pellets were resuspended with fresh medium (see 1c) and cells were counted using a hemocytometer after Trypan blue staining.
iii. Cells were seeded at the densities given in Table 4 in 100 uL media in five 96-well black plates and allowed to attach overnight.

TABLE 4
Number of cells from each cell line seeded
on Day 0 of proliferation assay.

| | Number of cells seeded per well |
|---|---|
| Parental cell line | on Day 0 of proliferation assay |
| HSC-2 | 250 |
| Daoy | 200 |
| SK-MEL-24 | 1000 |
| HCC1806 | 500 |
| NCI-H1563 | 1000 |

iv. The next day, 100 uL CTG/well was added to cell seeded plate. Plates were rocked for 10 min at RT to lyze cells and incubated at RT for 10 mins to stabilize luminescent signal. Luminescence was measured and this reading was set as day0.
v. Viable cell count was also measured by CTG assay on days 1, 3, 5 and 7. 100 ul/well fresh medium was added on Day 4.

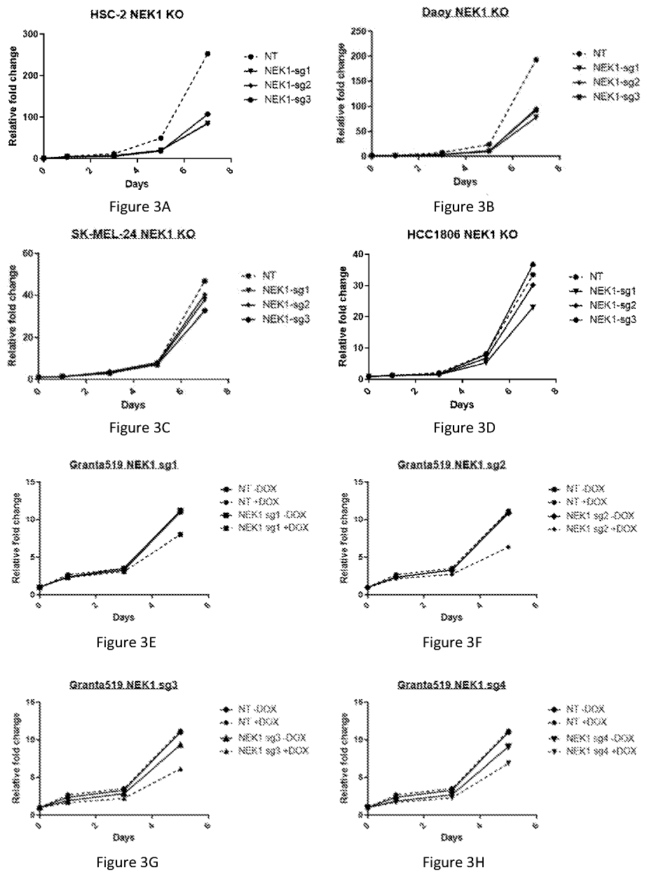

Growth curves were generated and normalised relative to the Day0 timepoint; these curves are shown in FIGS. 3A-D. These curves demonstrate that the NEK1 knockout had a more pronounced effect on HSC-2 and DAOY cell lines than the other cell lines tested.

### Example 4b—Cell Proliferation Assay—Inducible Knockout

i. Granta-519 cells treated with inducible lentiviral vectors (pRSGT16-U6Tet-puro) comprising sgNT and sg1-4 were grown in the presence or absence of doxycycline (1 mg/mL) for 7 days with a refresh of the medium on day 4.
ii. Cells were obtained by trypsinising adhered cells then centrifuging at 200 g for 5 minutes at room temperature (RT).
iii. Cell pellets were resuspended with fresh medium (see 1c) and Western blotting was used to detect the presence or absence of NEK-1 (FIG. 1F). Cells were then counted using a hemocytometer after Trypan blue staining.
iv. Cells were seeded at 7500 cells/well in 100 uL media in five 96-well black plates and allowed to attach overnight.
v. The next day, 100 uL CTG/well was added to cell seeded plate. Plates were rocked for 10 min at RT to lyze cells and incubated at RT for 10 mins to stabilize luminescent signal. Luminescence was measured and this reading was set as day0.
vi. Viable cell count was also measured by CTG assay on days 1, 3 and 5.

Growth curves were generated and normalised relative to the Day0 timepoint; these curves are shown in FIGS. 3E-H. These curves demonstrate that induction of the NEK-1 knockout inhibited cell growth.

### Example 5—Effect of ATM Inhibitor on NEK1 Knockouts

i. Adhered cells were washed with 1 mL DPBS and trypsinised by addition of trypsin to 0.05% w/v and incubation under standard conditions.
ii. 1 mL cell culture medium was added to the detached cells to inhibit trypsin and suspended cells were centrifuged at 200×g for 5 minutes at room temperature.
iii. The supernatant was discarded and the cells were resuspended in 1 ml cell culture medium and exact cell number was determined using hemocytometer after Trypan blue staining.
iv. Cells were seeded according to table 5 in 90 μl/well in opaque 96-well plates in triplicate. Cells were grown overnight under standard conditions in complete culture medium (see 1c) with drugs.

TABLE 5
Number of cells of each cell type seeded
on Day 0 of Compound profiling assay.

| | Number of cells seeded per well on Day 0 |
|---|---|
| Parental cell line | of ATM inhibition viability assay |
| HSC-2 | 250 |
| Daoy | 200 |
| HCC1806 | 500 |
| NCI-H1563 | 1000 |

v. Cells were treated with AZD1390 over the concentration range 10 μM-0.00152 μM, prepared by 10-point 3× serial dilutions from a 10 mM DMSO stock. Cells were incubated under standard conditions with the compounds for 7 days with refresh of the medium and drug twice a week.
vi. After the 7 day incubation, CGT assays were performed to assess cell viability.

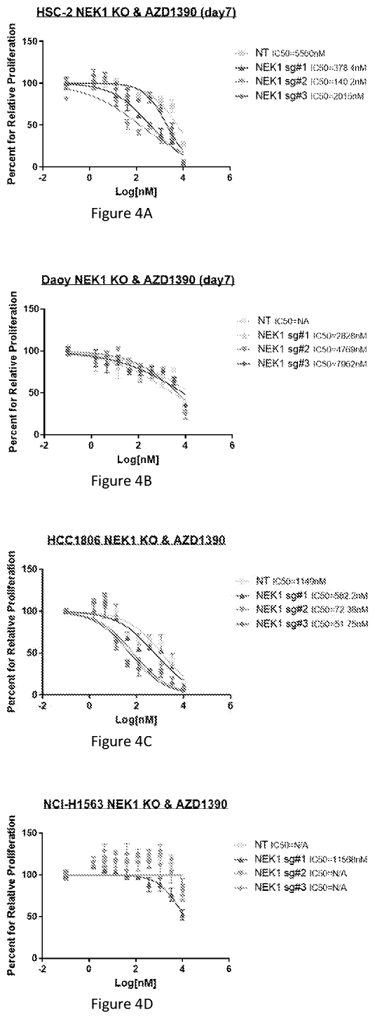

Data showing cell viability for NEK1 knockout cell lines and NT cell lines are shown in FIGS. 4A-D. Data are normalized relative to cells treated with DMSO only.

The data demonstrates that the ATM inhibitor had a synergistic effect on cell viability when combined with a NEK-1 knockout in HSC-2, Daoy and HCC1806 cells.

### Example 6—Evaluation of In Vivo Anti-Tumour Efficacy of NEK1 Gene in the Inducible HSC-2 NT and NEK1 KO-sq2 Xenograft Model in CB17 SCID Mice

Animals used:
Species: Mus musculus
Strain: CB17 SCID
Age: 6-8 weeks
Sex: female
Body weight: 18.78˜21.82 g
Number of animals: 50
Animal supplier: Beijing Vital River Laboratory Animal Technology Co., Ltd.

Animals were acclimatised for a period of approximately 3-7 days between arrival and tumor inoculation in order to accustom the animals to the laboratory environment. The mice were maintained in a special pathogen-free environment and in individual ventilation cages (5 mice per cage). All cages, bedding, and water were sterilized before use. When working in the mouse room, the investigators wore lab coat and latex or vinyl gloves. Each cage was clearly labelled with a cage card indicating number of animals, sex, strain, date received, treatment, study number, group number, and the starting date of the treatment. The cages with food and water were changed twice a week. The targeted conditions for animal room environment and photoperiod were as follows:
Temperature: 20-26° C.
Humidity 40-70%.
Cages: Made of polycarbonate. The size is 300 mm×180 mm×150 mm. The bedding material was corn cob, which was changed twice per week.
Diet: Animals had free access to irradiation sterilized dry granule food during the entire study period.
Water: Animals had free access to sterile drinking water.
Animal identification: Animals were marked by ear coding.

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). At the time of routine monitoring, the animals were daily checked for any effects of tumour growth and treatments on normal behaviour such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset.

HSC-2 NT and inducible HSC-2 NEK1 KO-sg2 cells (transfected with pRSGT16-U6Tet-puro vector system) were maintained in vitro as a monolayer culture in EMEM medium supplemented with 10% Tet-free FBS, 1 μg/mL puromycin and 5 μg/mL Blasticidin at 37° C. in an atmosphere of 5% CO₂ in air. The tumour cells were routinely subcultured twice weekly by trypsin-EDTA treatment.

Animals were assigned into groups using an Excel-based randomization software performing stratified randomization based upon their body weights. Each group consisted of 10 or 15 tumour-bearing mice (See Table 6). Cells growing in an exponential growth phase were harvested and counted for tumour inoculation. Each mouse was inoculated subcutaneously at the right flank with HSC-2 NT or HSC-2 NEK1 KO-sg2 cells (1×10⁶) in 0.2 mL of PBS with Matrigel (1:1) for tumour development. Dox chow (400 mg/kg) treatments were started right after tumour inoculation to induce NEK1 knock down.

TABLE 6
Animal groups with cell and treatment types

| | | Number of | |
|---|---|---|---|
| Group | Cell | animals | Dose |
| 1 | HSC-2 NT | 15 | −Dox |
| 2 | HSC-2 NT | 10 | +Dox |
| 3 | HSC-2 NEK1 KO-sg2 | 15 | −Dox |
| 4 | HSC-2 NEK1 KO-sg2 | 10 | +Dox |

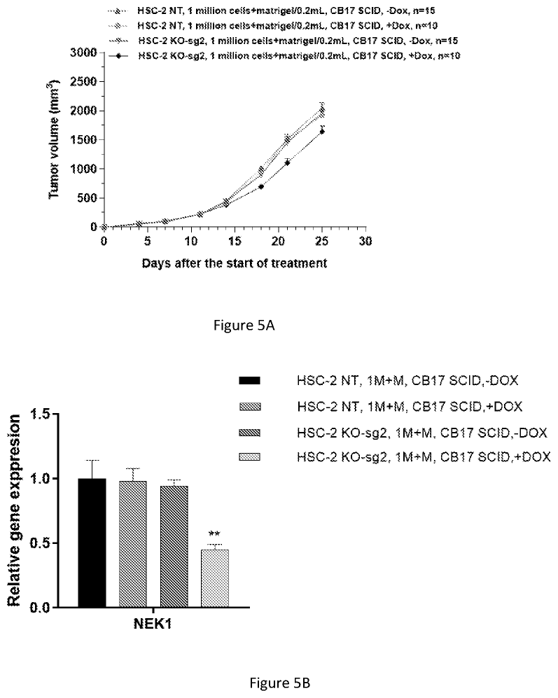

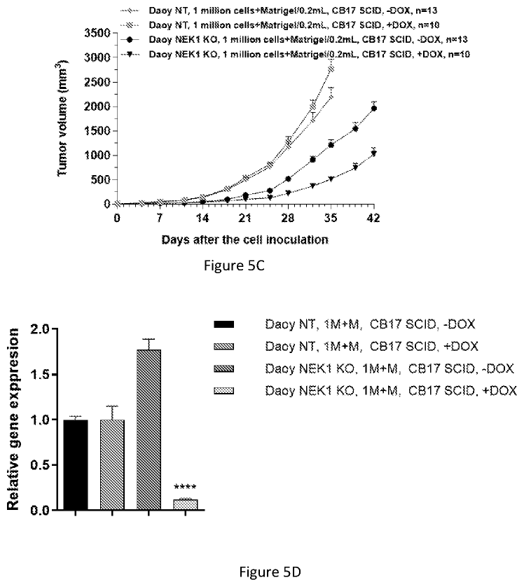

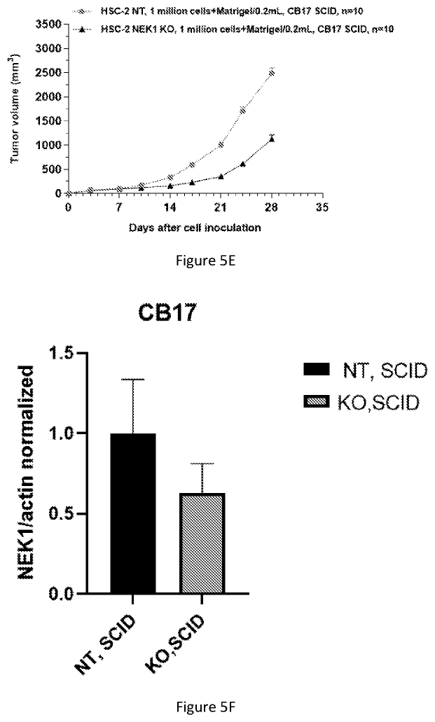

Tumour size was measured twice weekly in two dimensions using a calliper, and the volume was expressed in mm³ using the formula: V=0.5a×b² where a and b are the long and short diameters of the tumour, respectively. (See Table 7, FIG. 5A). A T-test was performed to 7 compare tumour volume at day 28 between Group 3 and Group 4. p<0.05 was considered to be statistically significant.

Tumour Growth Inhibition (TGI) was calculated using the formula:

$$TGI ( \% ) = [ 1 - ( T / V ) ] \times 100 wherein :$$

T is the average tumour volume of a treatment group on day 28, and
V is the average tumour volume of the vehicle control group on day 28

TABLE 7
Mean and standard deviation of tumour volumes for
each animal group. Day 0 represents inoculation.

| | Tumour volume (mm³) | | | |
|---|---|---|---|---|
| Day | Group 1 | Group 2 | Group 3 | Group 4 |
| 0 | 0 ± 0 | 0 ± 0 | 0 ± 0 | 0 ± 0 |
| 4 | 59 ± 2 | 55 ± 1 | 54 ± 2 | 63 ± 3 |
| 7 | 105 ± 5 | 97 ± 4 | 90 ± 3 | 95 ± 3 |
| 11 | 221 ± 8 | 228 ± 12 | 224 ± 11 | 226 ± 8 |
| 14 | 451 ± 18 | 422 ± 30 | 445 ± 27 | 381 ± 21 |
| 18 | 1014 ± 40 | 959 ± 50 | 901 ± 45 | 695 ± 41 |
| 21 | 1512 ± 81 | 1498 ± 64 | 1454 ± 95 | 1104 ± 78 |
| 25 | 2050 ± 91 | 1923 ± 98 | 1964 ± 154 | 1642 ± 97 |
| 28 | 2639 ± 154 | 2357 ± 100 | 2455 ± 206 | 2177 ± 105 |
| | | | | p = 0.2449 vs |
| | | | | Group 3 |
| | | TGI = 10.68 | | TGI = 11.34% |

Gene expression in tumour samples was quantified using qPCR. Expression of NEK1 was normalised to GAPDH expression (FIG. 5B).

NEK1 Forward Primer (SEQ ID NO. 6):
TGAAAAGTTTCTCTCTCCTCAG
NEK1 Reverse Primer (SEQ ID NO. 7):
AACAGAAATCGAGTTTTGTCCT
GAPDH Forward Primer (SEQ ID NO. 8):
TCAAGGCTGAGAACGGGAAG
GAPDH Reverse Primer (SEQ ID NO. 9):
CGCCCCACTTGATTTTGGAG

### Example 7 Evaluation of In Vivo Anti-Tumour Efficacy of NEK1 Gene in the Inducible Daoy NT and NEK1 KO Xenograft Model in CB17 SCID Mice

Animals used:
Species: Mus musculus
Strain: CB17 SCID
Age: 6-8 weeks
Sex: female
Body weight: 16.58˜20.51 g
Number of animals: 46
Animal supplier: Zhejiang Vital River Laboratory Animal Technology Co., Ltd.

Animals were acclimatised for a period of approximately 3-7 days between arrival and tumour inoculation in order to accustom the animals to the laboratory environment. The mice were maintained in a special pathogen-free environment and in individual ventilation cages (5 mice per cage). All cages, bedding, and water were sterilized before use. When working in the mouse room, the investigators wore lab coat and latex or vinyl gloves. Each cage was clearly labelled with a cage card indicating number of animals, sex, strain, date received, treatment, study number, group number, and the starting date of the treatment. The cages with food and water were changed twice a week. The targeted conditions for animal room environment and photoperiod were as follows:
Temperature: 20-26° C.
Humidity 40-70%.
Cages: Made of polycarbonate. The size is 300 mm×180 mm×150 mm. The bedding material was corn cob, which was changed twice per week.
Diet: Animals had free access to irradiation sterilized dry granule food during the entire study period.
Water: Animals had free access to sterile drinking water.
Animal identification: Animals were marked by ear coding.

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). At the time of routine monitoring, the animals were daily checked for any effects of tumour growth and treatments on normal behaviour such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset.

Daoy NT and inducible Daoy NEK1 KO-sg2 cells (transfected with pRSGT16-U6Tet-puro vector system) were maintained in vitro as a monolayer culture in EMEM medium supplemented with 10% Tet-free FBS, 1 μg/mL puromycin and 5 μg/mL Blasticidin at 37° C. in an atmosphere of 5% CO₂ in air. The tumor cells were routinely subcultured twice weekly by trypsin-EDTA treatment.

Animals were assigned into groups using an Excel-based randomization software performing stratified randomization based upon their body weights. Each group consisted of 10 or 13 tumour-bearing mice (See Table 8). Dox chow (400 mg/kg) treatments were started 7 days before inoculation to induce NEK1 knock down. Cells growing in an exponential growth phase were harvested and counted for tumour inoculation. Each mouse was inoculated subcutaneously at the right flank with Daoy NT or Daoy NEK1 KO-sg2 cells (1×10⁶) in 0.2 mL of PBS with Matrigel (1:1) for tumour development.

TABLE 8
Animal groups with cell and treatment types

| | | Number of | |
|---|---|---|---|
| Group | Cell | animals | Dose |
| 5 | Daoy NT | 13 | −Dox |
| 6 | Daoy NT | 10 | +Dox |
| 7 | Daoy NEK1 KO-sg2 | 13 | −Dox |
| 8 | Daoy NEK1 KO-sg2 | 10 | +Dox |

Tumour size was measured twice weekly in two dimensions using a calliper, and the volume was expressed in mm³ using the formula: V=0.5a×b² where a and b are the long and short diameters of the tumour, respectively (See table 9, FIG. 5B). A T-test was performed to compare tumour volume at day 42 between Group 7 and Group 8. p<0.05 was considered to be statistically significant.

Tumour Growth Inhibition (TGI) was calculated using the formula:

$$TGI ( \% ) = [ 1 - ( T / V ) ] \times 100 wherein :$$

T is the average tumour volume of the treatment group on day 42, and
V is the average tumour volume of the vehicle control group on day 42.

TABLE 9
Mean and standard deviation of tumour volumes for
each animal group. Day 0 represents inoculation.

| | Tumour volume (mm³) | | | |
|---|---|---|---|---|
| Day | Group 5 | Group 6 | Group 7 | Group 8 |
| 0 | 0 ± 0 | 0 ± 0 | 0 ± 0 | 0 ± 0 |
| 4 | 31 ± 2 | 29 ± 3 | 14 ± 2 | 13 ± 2 |
| 7 | 50 ± 3 | 50 ± 5 | 21 ± 3 | 17 ± 2 |
| 11 | 88 ± 3 | 84 ± 4 | 31 ± 4 | 28 ± 3 |
| 14 | 144 ± 10 | 148 ± 5 | 52 ± 7 | 44 ± 6 |
| 18 | 300 ± 37 | 314 ± 20 | 101 ± 10 | 71 ± 10 |
| 21 | 496 ± 55 | 543 ± 29 | 184 ± 17 | 102 ± 15 |
| 25 | 764 ± 76 | 818 ± 49 | 280 ± 26 | 128 ± 20 |
| 28 | 1171 ± 111 | 1285 ± 98 | 521 ± 49 | 225 ± 34 |
| 32 | 1713 ± 169 | 1997 ± 136 | 907 ± 76 | 371 ± 40 |
| 35 | 2193 ± 190 | 2764 ± 203 | 1213 ± 106 | 515 ± 48 |
| 39 | | | 1545 ± 124 | 747 ± 88 |
| 42 | | | 1961 ± 137 | 1025 ± 131 |
| | | | | p = 0.0002 vs |
| | | | | Group 7 |
| | | | | TGI = 47.69% |

Gene expression in tumour samples was quantified using qPCR. Expression of NEK1 was normalised to GAPDH expression (FIG. 5D).

NEK1 Forward Primer (SEQ ID NO. 6):
TGAAAAGTTTCTCTCTCCTCAG
NEK1 Reverse Primer (SEQ ID NO. 7):
AACAGAAATCGAGTTTTGTCCT
GAPDH Forward Primer (SEQ ID NO. 8):
TCAAGGCTGAGAACGGGAAG
GAPDH Reverse Primer (SEQ ID NO. 9):
CGCCCCACTTGATTTTGGAG

### Example 8 Evaluation of In Vivo Anti-Tumor Efficacy of NEK1 Gene in Constitutive HSC2 NT and NEK1 KO Xenograft Model in CB17 SCID Mice

Animals used:
Species: Mus musculus
Strain: CB17 SCID
Age: 6-8 weeks
Sex: female
Body weight: 18-22 g
Number of animals: 20
Animal supplier: Vital River Laboratory Animal Technology Co., LTD.

Animals were acclimatised for a period of approximately 7 days between arrival and tumour inoculation in order to accustom the animals to the laboratory environment. The mice were maintained in a special pathogen-free environment and in individual ventilation cages (5 mice per cage). All cages, bedding, and water were sterilized before use. When working in the mouse room, the investigators wore lab coat and latex or vinyl gloves. Each cage was clearly labelled with a cage card indicating number of animals, sex, strain, date received, treatment, study number, group number, and the starting date of the treatment. The cages with food and water were changed twice a week. The targeted conditions for animal room environment and photoperiod were as follows:
Temperature: 20-26° C.
Humidity 40-70%.
Cages: Made of polycarbonate. The size is 300 mm×180 mm×150 mm. The bedding material was corn cob, which was changed twice per week.
Diet: Animals had free access to irradiation sterilized dry granule food during the entire study period.
Water: Animals had free access to sterile drinking water.
Animal identification: Animals were marked by ear coding.

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). At the time of routine monitoring, the animals were daily checked for any effects of tumour growth and treatments on normal behaviour such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset.

The HSC2 NT and constitutive NEK1 KO-sg2 cells (transfected with LentiCRISPRv2-puro vector system) were maintained in vitro as a monolayer culture in EMEM medium supplemented with 10% Tet-free FBS, 1 μg/mL puromycin and 5 μg/mL Blasticidin at 37° C. in an atmosphere of 5% CO2 in air. The tumor cells were routinely subcultured twice weekly by trypsin-EDTA treatment.

Animals were assigned into groups using an Excel-based randomization software performing stratified randomization based upon their body weights. Each group consisted of 10 tumour-bearing mice (See Table 10). Cells growing in an exponential growth phase were harvested and counted for tumour inoculation. Each mouse was inoculated subcutaneously at the right flank with HSC2 NT or NEK1 KO cells (1×106) in 0.2 mL of PBS with Matrigel (1:1) for tumor development.

TABLE 10
Animal groups with cell types

| Group | Cell | Number of animals |
|---|---|---|
| 9 | HSC-2 NT cell | 10 |
| 10 | HSC-2 NEK1 KO cell | 10 |

Tumour size was measured twice weekly in two dimensions using a calliper, and the volume was expressed in mm³ using the formula: V=0.5a×b² where a and b are the long and short diameters of the tumour, respectively (See table 11, FIG. 5C). A T-test was performed to compare tumour volume at day 28 between Group 9 and Group 10. p<0.05 was considered to be statistically significant.

Tumour Growth Inhibition (TGI) was calculated using the formula:

$$TGI ( \% ) = [ 1 - ( T / V ) ] \times 100 wherein :$$

T is the average tumour volume of the treatment group on day 28, and
V is the average tumour volume of the vehicle control group on day 28

TABLE 11
Mean and standard deviation of tumour volumes for
each animal group. Day 0 represents inoculation.

Tumour volume (mm³)

| Day | Group 9 | Group 10 |
|---|---|---|
| 0 | 0 ± 0 | 0 ± 0 |
| 3 | 72 ± 5 | 59 ± 5 |
| 7 | 98 ± 6 | 93 ± 5 |
| 10 | 172 ± 12 | 116 ± 7 |
| 14 | 327 ± 24 | 160 ± 9 |
| 17 | 592 ± 41 | 229 ± 14 |
| 21 | 1003 ± 55 | 351 ± 31 |
| 24 | 1705 ± 87 | 614 ± 47 |
| 28 | 2486 ± 108 | 1124 ± 88 |
| | | p = 0.00028 vs Group 9 |
| | | TGI = 45.21% |

Gene expression in tumour samples was quantified using Western blotting. Expression of NEK1 was normalised to beta-actin expression (FIG. 5F).

1. A pharmaceutical composition for use in the treatment of head and neck cancer, mantle cell lymphoma or melanoma, wherein the composition comprises an NEK1 inhibitor.

2. The pharmaceutical composition for use of claim 1, wherein the composition is for use in the treatment of head and neck cancer.

3. The pharmaceutical composition for use of claim 1, wherein the composition is for use in the treatment of oral cancer.

4. The pharmaceutical composition for use of claim 1, wherein the composition is for use in the treatment of mantle cell lymphoma.

5. The pharmaceutical composition for use of claim 1, wherein the composition is for use in the treatment of melanoma.

6. A pharmaceutical combination for use in the treatment of cancer comprising the pharmaceutical composition of claim 1 and one or more additional therapeutic agents in synergistically effective amounts for simultaneous, separate or sequential administration to a subject in need thereof, wherein the one or more additional therapeutic agents comprise an ataxia telangiectasia mutated (ATM) kinase inhibitor.

7. The pharmaceutical combination for use of claim 6, wherein the ATM kinase inhibitor is one or more of AZD1390, AZ32, KU-55933, KU60019, KY12420, Torin2 or ETP46464.

8. The pharmaceutical combination for use of claim 6, wherein the ATM kinase inhibitor is AZD1390.

9. The pharmaceutical combination of claim 6, wherein the cancer is breast cancer.

10. The pharmaceutical combination of claim 6, wherein the cancer is oral cancer.

11. The pharmaceutical combination for use of claim 10, wherein the cancer exhibits a p53 mutation and a homologous recombination (HR) mutation.

12. The pharmaceutical combination for use of claim 10, wherein the cancer exhibits a p53 mutation and a homologous recombination (HR) deficiency.

13. The pharmaceutical combination for use of claim 11, wherein the cancer does not exhibit an ATM mutation.

14. The pharmaceutical combination for use in the treatment of cancer of claim 6, wherein the ratio of the NEK1 inhibitor to the ataxia telangiectasia mutated (ATM) kinase inhibitor is equal or less than 1000:1, 500:1, 100:1, 50:1, 20:1, 10:1, 5:1, 2:1, 1.5:1, 1:1, 1:1.5, 1:2, 1:5, 1:10, 1:20, 1:50, 1:100, 1:500, 1:1000.

15. A pharmaceutical composition for use in the treatment of a cancer in a patient wherein the cancer exhibits a p53 mutation, wherein the composition comprises an NEK1 inhibitor.

16. The pharmaceutical composition for use of claim 15, wherein cancer additionally exhibits a homologous recombination (HR) mutation.

17. The pharmaceutical composition for use of claim 15, wherein cancer additionally exhibits a homologous recombination (HR) deficiency.

18. The pharmaceutical composition for use of claim 1, wherein the composition comprises (i) 0.05 mg to 100 mg or (ii) 0.5 mg to 50 mg of an NEK1 inhibitor.

19. The pharmaceutical combination for use of claim 6, wherein the composition or combination comprises (i) 0.05 mg to 100 mg or 0.5 mg to 50 mg of an NEK1 inhibitor.

20. (canceled)

21. (canceled)

22. The pharmaceutical combination for use of claim 6, wherein the combination comprises (i) 0.05 mg to 100 mg, (ii) 0.1 mg to 50 mg, or (iii) 0.5 mg to 25 mg of an ATM inhibitor.

23. (canceled)

24. (canceled)

25. A pharmaceutical composition comprising an NEK1 inhibitor and an ATM inhibitor.

26. A method for the treatment of a cancer, which comprises administering a therapeutically effective amount of the pharmaceutical composition of claim 1 to a subject in need thereof, wherein the cancer is head and neck cancer, mantle cell lymphoma, or melanoma.

27. A method for the treatment of a cancer, which comprises administering a therapeutically effective amount of the pharmaceutical combination of claim 6 to a subject in need thereof, wherein the cancer is head and neck cancer, mantle cell lymphoma, or melanoma.

28. A method for the treatment of a cancer, which comprises administering a therapeutically effective amount of the pharmaceutical composition of claim 15 to a subject in need thereof, wherein the cancer is head and neck cancer, mantle cell lymphoma, or melanoma.

29. A method for the treatment of a cancer, which comprises administering a therapeutically effective amount of the pharmaceutical composition of claim 25 to a subject in need thereof, wherein the cancer is head and neck cancer, mantle cell lymphoma, or melanoma.


SEQUENCE LISTING (9 sequences)

SEQ ID NO: 1 - DNA, 20 bp, synthetic construct
  source 1..20: mol_type = other DNA
tgaggatcatgtcgagcgcc

SEQ ID NO: 2 - DNA, 20 bp, synthetic construct
  source 1..20: mol_type = other DNA
ataactgagacaccaaactg

SEQ ID NO: 3 - DNA, 20 bp, synthetic construct
  source 1..20: mol_type = other DNA
aaggaaaaagaacttacctg

SEQ ID NO: 4 - DNA, 20 bp, synthetic construct
  source 1..20: mol_type = other DNA
gaagtgaagaggctgacatg

SEQ ID NO: 5 - DNA, 20 bp, synthetic construct
  source 1..20: mol_type = other DNA
ggaagtggtgaagtaaaggt

SEQ ID NO: 6 - DNA, 22 bp, synthetic construct
  source 1..22: mol_type = other DNA
tgaaaagtttctctctcctcag

SEQ ID NO: 7 - DNA, 22 bp, synthetic construct
  source 1..22: mol_type = other DNA
aacagaaatcgagttttgtcct

SEQ ID NO: 8 - DNA, 20 bp, synthetic construct
  source 1..20: mol_type = other DNA
tcaaggctgagaacgggaag

SEQ ID NO: 9 - DNA, 20 bp, synthetic construct
  source 1..20: mol_type = other DNA
cgccccacttgattttggag